(19) United States
(12) Patent Application Publication
(10) Pub. No.: US 2026/0334878 A1
(43) Pub. Date: Oct. 29, 2026

(54) PRESSURE SENSOR APPARATUS AND SYSTEMS AND METHODS COMPRISING SAME

(71) Applicant: The United States Government as represented by the Department of Veterans Affairs, Washington, DC (US)

(72) Inventor: Punit Vaidya, Cleveland, OH (US)

(21) Appl. No.: 19/103,727

(22) PCT Filed: Aug. 16, 2023
(86) PCT No.: PCT/US2023/030329

§ 371 Date: Feb. 13, 2025

Related U.S. Application Data
(60) Provisional application No. 63/398,404, filed on Aug. 16, 2022.

(51) Int. Cl. A61B 5/00; A61B 5/145; A61M 5/172

(52) U.S. Cl. CPC A61B 5/6843; A61B 5/0022; A61B 5/14532; A61B 5/7405; A61B 5/7455; A61B 5/746; A61M 5/1723; A61B 2560/0209

(57) ABSTRACT

An apparatus has a pressure sensor that is configured to generate pressure measurements indicative of pressure applied to the apparatus along a first axis and a computing device in communication with the pressure sensor. The computing device is configured to receive, from the pressure sensor, a plurality of pressure measurements comprising at least a first pressure measurement and a second pressure measurement, wherein each pressure measurement of the plurality of pressure measurements is spaced from each other pressure measurement of the plurality of pressure measurements by a predetermined duration; compare the first and second pressure measurements to a first predetermined pressure threshold; and cause, if the first and second pressure measurements exceed the first predetermined pressure threshold, an alarm.

### CROSS-REFERENCE TO RELATED APPLICATION

This application claims priority to and the benefit of the filing date of U.S. Provisional Patent Application No. 63/398,404, filed Aug. 16, 2022, the entirety of which is hereby incorporated by reference herein.

### FIELD

This disclosure relates to apparatuses that detect pressure. The apparatus can be used, for example, to alert a user who is in an undesirable position. For example, the apparatus can be used in communication with a glucose monitor to alert the user or a remote computing device that the user is applying a pressure to the glucose monitor that will cause the glucose monitor to provide an incorrect reading.

### BACKGROUND

Detecting an improper position of an individual can be important for applications such as, for example, measuring glucose using a continuous glucose monitor. Referring to FIG. 1, a conventional continuous glucose monitor (CGM) 1 includes a sensor 2 that extends through skin 8 and into interstitial fluid 3 (rather than into blood vessels 4). The sensor 2 senses glucose 5 that is exterior to cells 6. The conventional CGM has a transmitter for outputting glucose data to a smartphone or other connected apparatus. Pressure applied to the continuous glucose monitor 1 can cause the CGM to produce erroneous readings (typically, false hypoglycemic readings). This can be common during sleep, when the user inadvertently rolls over on top of the CGM. The erroneous readings can trigger alarms that wake up the user or a caretaker of the user (often, a parent). Further, the erroneous readings limit the ability of CGMs to be used with insulin pumps, as use of insulin pumps requires accuracy. For a minority of GCMs, covers can be worn over the CGM in order to prevent pressure being applied directly to the CGM, thereby reducing pressure artifacts. However, such covers are suitable only for extremities, and cannot be positioned on the abdomen or other non-extremity sites. The majority of CGMs, including the Dexcom G6, require the user to wear the CGM on the trunk. For this reason, covers are not a suitable solution for most users, which includes children with type 1 diabetes. Covers that protect on extremity placement sites are unwieldy, and may be uncomfortable or disruptive to the user.

Similarly, preventing improper positioning of an individual can be important in other circumstances. For example, improper positioning can lead to pressure ulcers. As another example, supine sleep position is associated with a 2.3× risk of late pregnancy stillbirth and other fetal complications, and pregnant women are encouraged to sleep on their left side. As yet another example, it can be beneficial to avoid application of pressure to a wound.

Thus, a need exists for detecting improper positioning of an individual and providing an alarm or other notification or output in response to the improper positioning.

### SUMMARY

Disclosed herein, in one aspect, is an apparatus comprising a pressure sensor that is configured to generate pressure measurements indicative of pressure applied to the apparatus along a first axis. A computing device is in communication with the pressure sensor, the computing device has a memory and at least one processor in communication with the memory. The memory has instructions that, when executed by the at least one processor, cause the at least one processor to: (a) receive, from the pressure sensor, a plurality of pressure measurements comprising at least a first pressure measurement and a second pressure measurement, wherein each pressure measurement of the plurality of pressure measurements is spaced from each other pressure measurement of the plurality of pressure measurements by a predetermined duration; (b) compare the first and second pressure measurements to a first predetermined pressure threshold; and cause, if the first and second pressure measurements exceed the first predetermined pressure threshold, an alarm.

In one aspect, a system comprises an apparatus as disclosed and a continuous glucose monitor comprising a sensor that is configured to extend along a first axis into subcutaneous fatty tissue of a user. The sensor is configured to determine a glucose measurement.

In one aspect, a system comprises an apparatus as disclosed, the apparatus comprising a transmitter. A remote computing device is in communication with the transmitter.

In one aspect, an apparatus includes a circuit having a pressure sensing device, a power source, and a switching device having a voltage threshold and switchable flow path.

The switching device is configured to permit current to flow to through a switchable current flow path upon receiving an applied voltage that exceeds the voltage threshold. An alarm is in communication with the switchable current flow path of the switching device. The pressure sensing device, the power source, the switching device, and the alarm are electrically coupled so that closing of the momentary switch causes power to flow from the power source to the capacitor to charge the capacitor. A voltage across the capacitor is configured to exceed the voltage threshold of the switching device after a predetermined time, thereby triggering the alarm.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a schematic diagram of a conventional continuous glucose monitor (CGM).

FIG. 2 is a schematic diagram of an apparatus as disclosed herein.

FIG. 3 is a schematic diagram of an exemplary system comprising the apparatus of FIG. 2.

FIG. 4 is another schematic diagram of an exemplary system comprising the apparatus of FIG. 2.

FIG. 5 is perspective view of an exemplary apparatus without a computing device.

FIG. 6 is a schematic diagram of a circuit of the apparatus of FIG. 5.

FIG. 7 is a schematic diagram of a circuit of an exemplary apparatus without a computing device.

FIG. 8 is a plot of a force vs resistance curve for a force sensing resistor of the apparatus of FIG. 7.

FIG. 9 is a top plan view of force sensing resistors for use with the apparatus of FIG. 7.

FIG. 10 is a block diagram of a computing system comprising a computing device in accordance with embodiments disclosed herein.

FIG. 11 is a perspective view of an exemplary apparatus as disclosed herein with an accelerometer exploded therefrom.

FIG. 12 is a front view of an exemplary apparatus as disclosed herein.

FIG. 13 is a side view of the exemplary apparatus of FIG. 12.

FIG. 14 is a front view of the exemplary apparatus of FIG. 12, rotated 90 degrees to show activation of an LED indicating a triggering orientation.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” can include plural references unless the context clearly dictates otherwise. Thus, for example, reference to “an alarm” includes both disclosure of one alarm and disclosure of more than one alarm, and so forth. Accordingly, except where the context clearly indicates otherwise, when a singular form of an element is disclosed, it is understood that the application provides support for embodiments in which only one of such elements is provided, as well as support for embodiments in which a plurality of such elements is provided.

As used herein “or” should be understood to be an inclusive or unless context dictates otherwise. For example, when separating items in a list, “or” should be interpreted as being inclusive, i.e., the inclusion of at least one, but also including more than one, of a number or list of elements, and, optionally, additional unlisted items. Thus, except where the context clearly dictates otherwise, the term “or” should be understood to provide support for embodiments that include only a single element of a list of elements, as well as for embodiments that include more than one of the elements from the list of elements.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step. In the preceding and following disclosure and claims, where embodiments including various elements are described using the word “comprise” or “comprising,” it is understood that the disclosure also includes embodiments that “consist of” or “consist essentially of” the same elements.

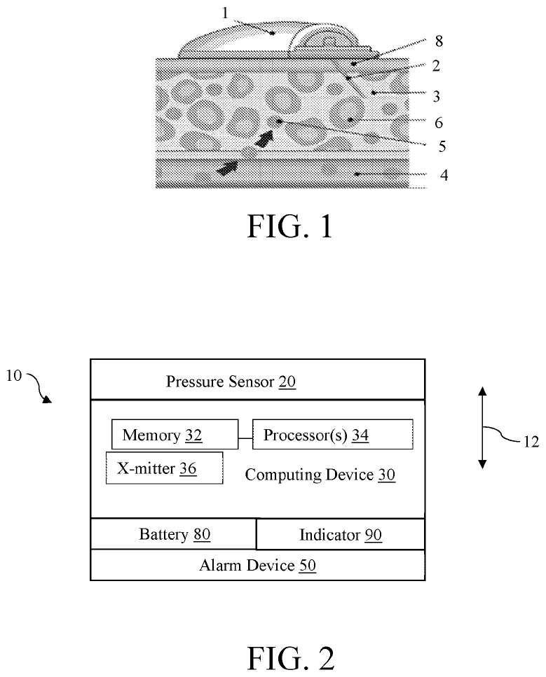

Disclosed herein, and with reference to FIGS. 1-3, is an apparatus 10 that can be used to determine when a user is in an undesirable position. The apparatus 10 can comprise a pressure sensor 20 that is configured to generate pressure measurements indicative of pressure applied to the apparatus along a first axis 12. Optionally, the apparatus 10 can comprise a plurality of pressure sensors 20. However, in some aspects, the apparatus 10 can include only a single pressure sensor 20. The apparatus 10 can further comprise a computing device 30 in communication with the pressure sensor 20. The computing device 30 can comprise a memory 32 and at least one processor 34 in communication with the memory. In exemplary aspects, the computing device 30 can be a microcontroller (e.g., optionally, a SEEEDUINO board manufactured by Seeed Studio (San Leandro, California)). In further aspects, it is contemplated that other microcontrollers can be used. For example, a microcontroller can have deep sleep capabilities, and an analog-to-digital convertor can be used. Some examples of suitable microcontrollers include a SAMD51 microcontroller, an ATTINY85 microcontroller, an Atmega328 microcontroller, an ESP32 microcontroller, which can provide WiFi and Bluetooth communication, and a Nordic nRF52840 microcontroller, which can provide Bluetooth Low Energy (BLE) communication.

The memory 32 can comprise instructions that, when executed by the at least one processor 34, cause the at least one processor to receive, from the pressure sensor 20, a plurality of pressure measurements comprising at least a first pressure measurement and a second pressure measurement. Each pressure measurement of the plurality of pressure measurements can be spaced from each other pressure measurement of the plurality of pressure measurements by a predetermined duration. The memory 32 can comprise further instructions that, when executed by the at least one processor, cause the at least one processor to (a) compare the first and second pressure measurements to a first predetermined pressure threshold; and (b) cause, if the first and second pressure measurements exceed the first predetermined pressure threshold, an alarm. Exceeding the first predetermined threshold can correspond to a portion of a bodyweight of a user resting on the pressure sensor 20. For example, the first predetermined threshold can correspond to a patient rolling on top of, or otherwise applying pressure to, the pressure sensor 20. Undesirable pressure can be caused by, for example, an individual sleeping on top of the pressure sensor 20 or leaning or pressing the pressure sensor against a surface (e.g., an arm rest or wall). It is contemplated that at least two pressure measurements above the first predetermined threshold can correspond to sustained positioning of the user on the pressure sensor 20.

It is contemplated that the apparatus 10 can be configured to have a low power usage in order to extend battery life. For example, during intervals between taking pressure measurements and associated processing of the pressure measurements (e.g., during the predetermined duration), the computing device 30 can operate in a low power mode. Optionally, in this low power mode, for example, the computing device can perform no processing other than operating a timer counting down to a next function or action.

In further aspects, certain functions can be carried out while the microcontroller is in low power mode. For example, certain functions can be programmed into the microcontroller, with some variation depending on the specific microcontroller that is used (e.g., by shutting off a USB driver, analog to digital converters, digital to analog converters, monitoring interrupt pins, toggling WiFi/BT/radio power, etc.). Peripheral sensor integrated circuits connected to power rails of the microcontroller can optionally remain active in low-power state and be configured to send an interrupt signal to wake up the MCU in certain conditions, such as upon the pressure sensor being pressed, or the pressure sensor being pressed for a predetermined duration.

Accordingly, in some aspects, the memory 32 of the computing device 30 can comprise instructions that, when executed by the at least one processor 34, cause the at least one processor to: cause the computing device to operate at a low power mode for an interval between receiving the first pressure measurement and receiving the second pressure measurement. In some optional aspects, the interval can be, or can substantially be, the predetermined duration between sequential pressure measurements. In some optional aspects, the interval (and, thus, the predetermined duration) can be from about 3 to about 30 seconds, or from about 5 seconds to about 15 seconds, or about 8 seconds. The interval can be selected based on the application for which the apparatus 10 is to be used.

It is contemplated that the apparatus 10 can be configured to detect sustained pressure on the apparatus for a certain/defined period of time. For example, the apparatus 10 can have a counter that counts a number of sequential pressure measurements that correspond to improper positioning, wherein the sequential pressure measurements are spaced over time and cumulatively correspond to said certain/defined period of time. In this way, brief, inconsequential durations of pressure on the apparatus do not trigger an alarm. If the user moves to remove the pressure so that the pressure sensor detects a pressure below the first threshold, the counter can reset.

Accordingly, in some aspects, the plurality of pressure measurements can comprise at least one additional pressure measurement, wherein the memory 32 of the computing device 30 comprises instructions that, when executed by the at least one processor 34, cause the at least one processor to: a) receive each pressure measurement of the at least one additional pressure measurement from the pressure sensor 20; b) compare each pressure measurement of the at least one additional pressure measurement to the first predetermined pressure threshold; c) cause a counter to increase for each measurement of the plurality of pressure measurements that exceeds the first predetermined pressure threshold; and d) cause the alarm only if the count exceeds a count threshold. In further aspects, the memory 32 of the computing device 30 comprises instructions that, when executed by the at least one processor 34, cause the at least one processor to: reset the counter when each measurement of the plurality of measurements from the pressure sensor 20 is below the first threshold.

The count threshold can correspond to a continued pressure above the first predetermined pressure threshold for a threshold duration. For example, the threshold duration can be at least 20 seconds, or at least 30 seconds or at least one minute, or no greater than 10 minutes, or no greater than 5 minutes, or no greater than 2 minutes, or no greater than one minute, or from about 20 seconds to about 10 minutes, or from about 30 seconds to about 5 minutes, or from about 30 seconds to about 2 minutes, or from about 30 seconds to about one minute. Thus, for example, a count of four with an interval of 8 seconds between pressure measurements can correspond to a threshold duration of 24 seconds. In other aspects, to prevent ulcers, it can be advantageous for an individual to move about every few hours. Accordingly, in some aspects, the threshold duration can be from about 1 hour to about 4 hours, or about 2 hours. In yet other aspects, the threshold duration can be from about 10 minutes to about 1 hour, or about 15 minutes to about 30 minutes, or about 20 minutes.

Optionally, the microcontroller can be configured so that it is in low power mode until an external trigger wakes the microcontroller. The external trigger can be, for example, a digital high or low signal, change in signal, or analog signal above or below a certain threshold. The external signal can be referred to as an interrupt that causes the microcontroller to stop its current operations, whether awake or asleep, and perform operations in accordance with a set of instructions.

In some aspects, the microcontroller can stay in sleep/low power mode indefinitely and wake up upon receiving a signal from a pressure sensor (e.g., from a force sensing resistor input, mechanical switch, or analog accelerometer reading of the z-axis) crossing a threshold (due to applied pressure above a certain threshold, or positioning the device face down). Upon receiving such a signal from the pressure sensor (or accelerometer), the microcontroller can wake up from low power mode record the pressure reading (or accelerometer output), and increment the counter. For example, the counter can increase by 1, thereby tracking sustained pressure on the pressure sensor so that when the counter reaches a predetermined threshold, corresponding to a threshold time of sustained pressure, the microcontroller can be configured to cause the alarm. Optionally, the microcontroller can then go back to sleep (this time, for some programmed interval of seconds, minutes, or longer), wake up after the timer, read the pressure (or accelerometer output), increment the counter, etc., as with the initial prototype. However, if the signal is low (pressure no longer present above the threshold), the microcontroller can reset the counter, go back to sleep for an indefinite duration, and wake up the next time there is a pressure event. Such a configuration can eliminate the device having to wake up from low power mode. Rather, the microcontroller can wake up from low power mode when there is an event and allow the microcontroller to be in low power mode for longer periods.

The apparatus 10 can be particularly advantageously used during sleep. A balance can be struck between generating an alarm too soon, thereby unnecessarily interrupting sleep when the user may naturally move on her own, and generating an alarm too late, after the user has fallen into deeper sleep, when alarming is more disruptive or may not wake the user. For example, the count threshold can be selected to provide a threshold duration that is long enough to avoid false or unnecessary alarms while still providing an alarm to trigger repositioning during microarousal episodes during sleep.

In other aspects, as further disclosed herein, the apparatus 10 can be used with a glucose monitor having a sampling interval. The threshold duration (before triggering an alarm) can be selected to not exceed a certain number of inaccurate readings. Thus, for a glucose monitor having a sampling interval of one minute (taking readings every minute), the threshold duration can be set at about 1 minute or less; for a glucose monitor having a sampling interval of five minutes (taking readings every five minutes), the threshold duration can be set at about 5 minutes or less.

In further aspects, the apparatus 10 can use pressure measurements of the pressure sensor 20 as an input device. For example, a user intentionally pressing on the pressure sensor 20 can generate a substantially higher pressure than an inadvertent pressure from rolling on top of the pressure sensor 20. Accordingly, in some aspects, the memory 32 of the computing device 30 can comprise instructions that, when executed by the at least one processor 34, cause the at least one processor to: compare the first pressure measurement to a second predetermined pressure threshold, wherein the second predetermined pressure threshold is greater than the first predetermined pressure threshold and is indicative of an intentionally applied pressure; and initiate a function if the first measurement is greater than the second predetermined pressure threshold. The function initiated can be, for example, a powering off of the apparatus 10 or a transition to stand-by mode. In stand-by mode, the apparatus 10 can, for example, have a lower power use. For example, in stand-by mode, the alarm can be disabled. In some aspects, in stand-by mode, the current draw can be down to a few microamps (for example, three microamps or less). For example, optionally, all functionality of the device except for internal oscillator timing can be disabled. The microcontroller can be preprogrammed with very basic instructions to wake up and do a task before entering deep sleep again. In exemplary aspects, the task can be to check for pressure, make note of the state of pressure, and then go back to sleep. In further aspects, the function initiated can be an adjustment to a setting of the device like changing the threshold time for the alarm, disabling the alarm, toggling power to transceiver, etc., depending on how the apparatus is programmed. An LED, piezo buzzer tone sequence, and/or haptic feedback (like a certain number of rapid pulses of the motor) can be used to indicate to the user when the device settings have changed.

In further aspects, the function can be initiated only if the user maintains pressure applied to the pressure sensor 20 for a predetermined duration. In this way, inadvertent initiation of the function can be avoided. For example, the memory of the computing device 30 can comprise instructions that, when executed by the at least one processor 34, cause the at least one processor to: compare the second pressure measurement to the second predetermined pressure threshold; and initiate the function only if the first and second pressure measurements are above the second predetermined pressure threshold.

It should be understood that in aspects in which the apparatus 10 is configured to compare pressure measurements to a second predetermined pressure that is greater than the first predetermined pressure (e.g., an intentional application of pressure by the user), the apparatus does not count a measured pressure above the second predetermined pressure as improper positioning. Accordingly, the memory 32 of the computing device 30 can comprise instructions that, when executed by the at least one processor 34, cause the at least one processor to cause the counter not to increase (to remain at the current count) for each measurement of the plurality of pressure measurements that exceeds the second predetermined pressure threshold.

The apparatus can comprise an alarm device 50. The alarm device 50 can be configured to generate, an audible alarm, a haptic/tactile alarm (e.g., a vibratory alarm), a visible alarm (e.g., a light that can optionally flash), an electrical stimulation alarm, or any other suitable alarm. Thus, it is contemplated that the alarm device 50 can comprise a speaker or other audible alarm device, a vibration alarm device, a haptic alarm device, a tactile alarm device, a visible alarm device (e.g., a light, a diode, a projector, and the like), a stimulating electrode, or the like. Optionally, the audible alarm can comprise a voice recording. For example, the voice recording can be that of a voice that is familiar to the user. For example, a parent or guardian can provide a voice recording for a child. It is contemplated that a voice recording of a familiar voice (e.g., a selected voice recording) can more effectively and more gently wake the user or encourage the user to reposition (optionally, without fully waking the user).

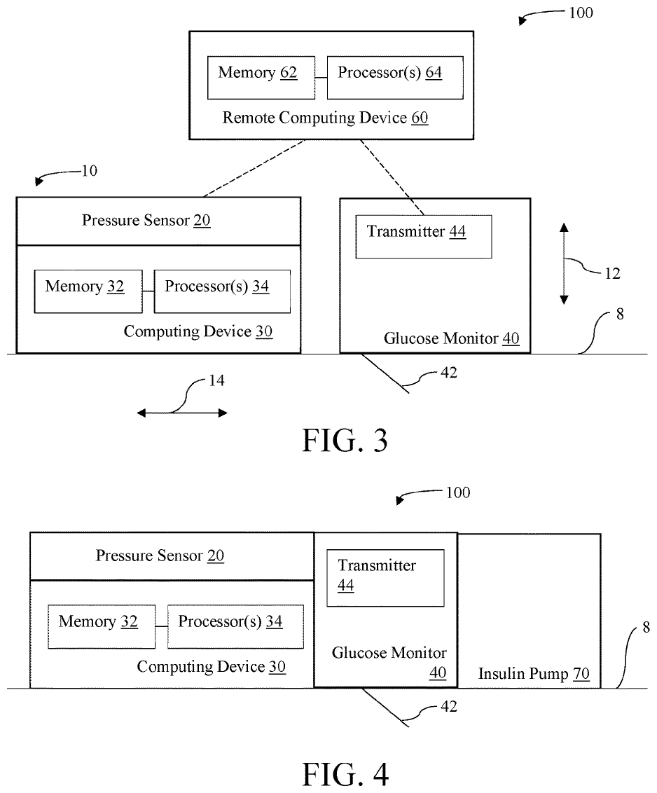

Referring to FIGS. 3 and 4, in some aspects, a system 100 can comprise a glucose monitor 40 (e.g., a continuous glucose monitor) having a sensor 42 that is configured to extend along a first axis 12 into subcutaneous fatty tissue of a user. The sensor 42 can be configured to determine a glucose measurement. Optionally, the glucose monitor 40 can be integral to the apparatus 10. In other aspects, the glucose monitor 40 can be a separate component from the apparatus 10.

In some aspects, the pressure sensor 20 can be positioned laterally of the glucose monitor 40 along a lateral axis 14 that is perpendicular to the first axis 12. For example, the pressure sensor can be immediately adjacent the glucose monitor 40. In this way, compression of the pressure sensor 20 likely corresponds to compression of the glucose monitor 40. In these aspects, the apparatus 10 can have approximately the same height along the first axis 12 as the glucose monitor 40 (e.g., within 25%, or 20%, or 15%, or 10%, or 5% of the height of the glucose monitor). In other aspects, the pressure sensor 20 can be laterally spaced from the glucose monitor 40 along the lateral axis 14. In still other aspects, the pressure sensor can be positioned above (away from the skin) or below (toward the skin) the continuous glucose monitor 40 along the first axis 12.

Referring to FIG. 4, the system 100 can further comprise an insulin pump 70 in communication with the glucose monitor 40. The insulin pump 70 can be configured to deliver insulin based on glucose measurements received from the glucose sensor 42. The insulin pump 70 can be regulated by one or more algorithms, including, for example, CONTROL IQ, by TANDEM (on a T-SLIM pump), or OMNIPOD 5 (by INSULET). In other aspects, the insulin pump 70 can be regulated by one or more open-sourced algorithms, such as, for example, LOOP or ANDROID APS, which can be compatible with various brands of CGMs and pumps.

In some optional aspects, the insulin pump 70 can be in communication with the computing device 30 of the apparatus 10. The insulin pump 70 can account for delivery of glucose by the insulin pump if the first pressure measurement or the second pressure measurement exceeds the first predetermined pressure threshold. For example, optionally, the computing device 30 can control insulin delivery of the insulin pump 70, and the computing device 30 can adjust insulin delivery based on pressure measurements from the pressure sensor 20.

In some aspects, the pressure sensor 20 can comprise one of a momentary switch, a barometric pressure sensor, a piezoelectric sensor, a capacitive force sensor, a strain gauge, or a force-sensing resistor. In these aspects, the pressure sensor 20 can directly detect pressure applied to the apparatus 10. In alternative aspects, the pressure sensor 20 can comprise an accelerometer that can detect undesirable positioning, thereby indirectly measuring pressure. For example, the accelerometer can measure orientation of the apparatus 10 (and, thus, infer pressure applied). It can be inferred that the accelerometer detecting an acceleration of about −1 g (i.e., −9.8 m/s²) along the first axis can correspond to a user lying in a particular orientation (e.g., on her back).

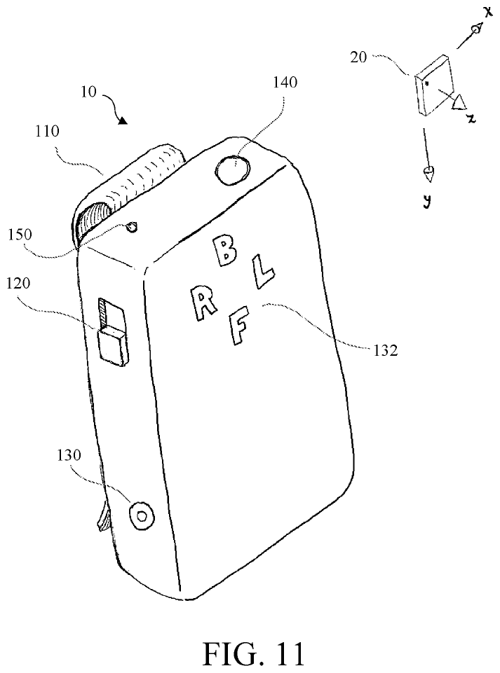

FIG. 11, shows an embodiment of the apparatus 10 that uses an accelerometer as the pressure sensor 20. The accelerometer readings can be used to detect orientation. In some aspects, the orientation can correspond to compression artifacts in a CGM. For example, a particular orientation can correspond to an individual lying on the CGM. In other aspects, the orientation can correspond to undesirable orientations of a user, such as, for example, during later stages of pregnancy or for wound care or pressure ulcers. In some aspects, the apparatus 10 need not be mounted directly over the skin or on the surrounding adhesive of the CGM. For example, the apparatus 10 can be attached centrally to the waistband, using a clip 110. The pressure sensor 20 is shown exploded from the apparatus 10, embodied as an integrated circuit (IC) accelerometer. The computing device 30 (FIG. 2) in the device can poll the accelerometer on a regular basis to determine the orientation of the wearer. In one example, if the wearer is lying flat (e.g., as during sleep, when compressions are more likely to occur), the y-axis reading from the accelerometer can be around 0 G (+/− some tolerance value, such as, for example, 0.15 G). If turned to the left, x-axis readings can be around 1 G. If turned to the right, x-axis readings can be around −1 G. If turned to the front, z-axis readings can be around −1 G. The apparatus 10 can include a power switch 120. In some aspects, the apparatus can comprise an input device (e.g., a recessed tactile switch or a slide switch) for toggling the alarm orientation setting (e.g., which orientation triggers an alarm). In this way, the orientation of the device can be selected to accommodate different wearing positions of the apparatus 10 or to accommodate different positions for the user to avoid. The apparatus 10 can further comprise a user feedback device 132 that indicates the orientation setting. For example, the output can comprise a plurality of light emitting diodes (LEDs). One of the LEDs corresponding to the different orientation setting (left, right, front, and back, corresponding to the wearer's perspective) can illuminate to indicate the current orientation setting and can be visible through translucent or transparent material formed as part of the housing. The LEDs can use low current, and also serve to indicate that the device is powered on. The user feedback device 132 (e.g., one or more of the LEDs) can be programmed to flash when the battery is low. When an alarm is triggered, a vibratory motor (e.g., an eccentric rotating mass (ERM) type or linear resonance actuator (LRA) type) can pulsate. In some aspects, the vibratory motor can pulsate with increasing intensity over time.

Optionally, another LED 140 and audible alarm can be triggered. A sound port 150 can permit a small speaker in the device to be audible. If the wearer does not change orientation in response to an alarm after a certain period, the computing device 30 (e.g., a microcontroller) can send another alert (e.g., via Bluetooth or Wi-Fi) to a remote device. The apparatus 10 can comprise a charging port and charging indicator LED (not shown in FIG. 11).

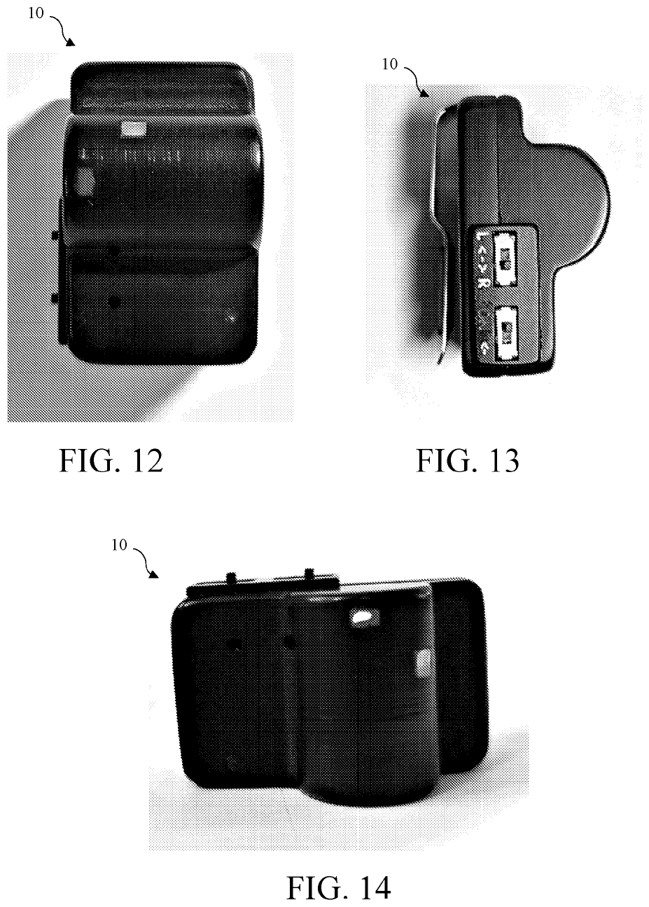

Referring to FIGS. 12-14, an exemplary apparatus 10 can include a green LED that is visible to indicate the device is turned on. The device can include the power switch and a slide switch used to set the orientation that is to be avoided (and that triggers an alarm). Note that in this exemplary apparatus, left and right are available options for the orientation setting. FIG. 14 shows the apparatus 10 turned to its left (and the slide switch is also set to left). The LED on top is illuminated to indicate and alarm is active, whereas, as shown in FIG. 12 with the apparatus 10 in a different orientation, the same LED is not illuminated. The apparatus can include a charging port and corresponding LED on the side of the device opposite that shown in FIG. 13. In some aspects, an LRA type vibratory motor and a custom-made circuit board can permit a smaller device size than that shown in FIGS. 12-14.

Referring to FIGS. 2-3, the apparatus 10 can comprise a transmitter 36 (e.g., a transceiver) that is configured to communicate with a remote computing device 60 having a memory 62 and at least one processor 64 in communication with the memory 62. The remote computing device 60 can be, for example, a smartphone. In other aspects, the remote computing device can be, for example, a smartwatch, a device by, under, or within the bed of the user that can be configured to provide a vibratory, audible, or other alarm (for example, a smart speaker or light (e.g., a smart bulb)). That is, the apparatus 10 can be configured to communicate with any suitable device that can communicate with the user or other party contributing to care of the user. In some aspects, the alarm can be provided by the remote computing device 60. For example, optionally, the apparatus 10 need not have an alarm device 50. Rather, the remote computing device 60 can be used to provide the alarm using an alarm device associated with the remote computing device. In some aspects, the apparatus 10 can trigger both an integral alarm device 50 and an alarm of a remote computing device 60.

In some aspects, the transmitter 36 can be a transceiver. In these aspects, the transceiver can be configured to receive a signal from the remote computing device 60, for example, to trigger an alarm to the apparatus 10 or adjust its settings.

In some optional aspects, the remote computing device 60 can comprise a transceiver that is configured to send data to a cloud-based device or network (or other computing device or network), from which an additional alarm can be triggered. For example, the remote computing device 60 can cause an internet of things (IoT) service to poll the online data that was uploaded by the remote computing device and send a text message alarm, phone call, or other alarm when an alarm condition is met.

In exemplary aspects, the apparatus 10 can send a transmission to the remote computing device 60 using very low power long range radio (e.g., LoRa) when an alarm condition is met. With LoRa, the apparatus 10 and the remote computing device 60 can be much farther away as compared to Bluetooth communication (e.g., up to kilometers away in ideal conditions). Upon receiving an alarm transmission from the apparatus, the remote computing device 60 can make an alarm sound, change a light or other visual output (e.g., flash an RGB LED red), and/or use a second (WiFi) radio to push this data to the cloud. The remote computing device 60 can subsequently send an automated phone call and/or text message to a caregiver and/or generate a notification through an application. Such an embodiment can be useful in cases where the user is an infant, elderly person, or may not have the wherewithal to reposition during an alarm.

In addition to the transmission sent by the apparatus 10 upon an alarm condition, the apparatus can also send packets of data to the remote computing device 60 in the background at a programmed interval (e.g., every 10 minutes), and also any time the pressure sensor detects a pressure exceeding a certain threshold (using an interrupt pin that wakes the microcontroller from sleep). The transmission can include one or more of the following data: device ID, battery level, temperature, raw pressure reading from the pressure sensor, how many seconds the pressure sensor has been in a pressed state (exceeding the second threshold), packet number, radio signal strength, and/or error code (for diagnostic purposes). Other data, like accelerometer data, can be included. A device identifier piece of data can further be provided in the transmission. Such data can be beneficial when multiple sensor devices are being monitored by one remote computing device, such as, for example, in a hospital setting. In some optional aspects, the remote computing device 60 can have a graphic display to show all or portions of the data sent by the sensor device, along with graphs of the historic data.

In some optional aspects, the apparatus 10 can be hermetically sealed. The apparatus 10 can comprise a battery 80. In some aspects, the battery 80 can be rechargeable (e.g., via wired or wireless charging). In other aspects, the battery 80 or, optionally, the apparatus 10, can be disposable. In some aspects, the battery 80 can have a battery life that is equal to or greater than the life or battery life of the glucose monitor (e.g., 7-14 days).

Optionally, the apparatus 10 can comprise an indicator 90 (e.g., a light, a buzzer, or a haptic motor). In some aspects, the alarm device 50 can serve as the indicator 90. In other aspects, the indicator 90 can be separate from the alarm device 50. The indicator 90 can provide various information including, for example, changing from stand-by mode to use (non-stand-by) mode. This information can be communicated, for example, by the indicator 90 turning on or off, flashing a pattern and/or changing color. Any combination of LED flashing/changing color, and/or haptic feedback from the motor can be used to convey various information, such as, for example, low battery, threshold level used to determine elevated pressure (e.g., low, medium, high sensitivity for different application), or if the transceiver is on or off, etc.

### Low Power Apparatus

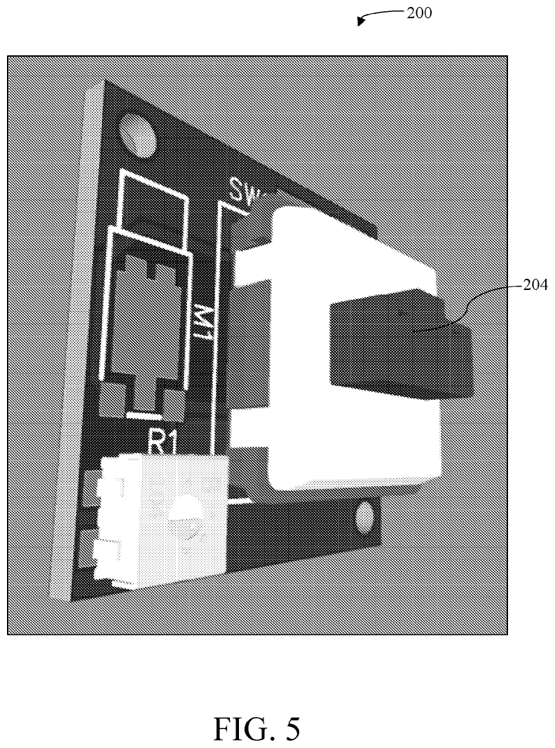

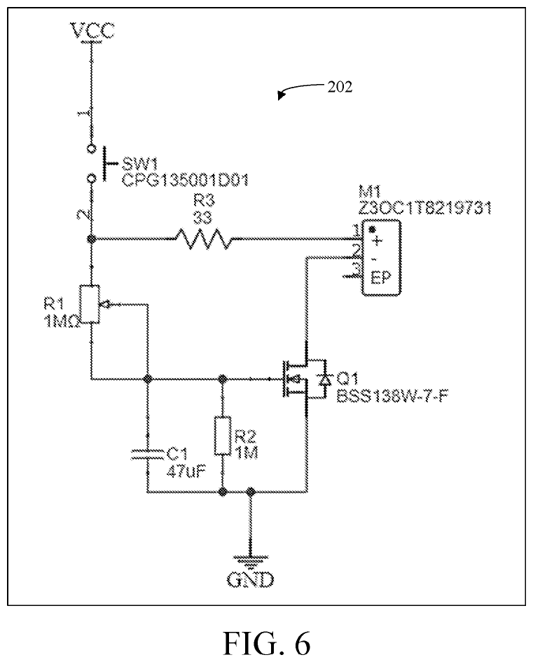

Referring to FIGS. 5-6, in some aspects, an apparatus 200 can detect applied pressure and trigger an alarm after a predetermined time without use of a microcontroller or other computing device. In this way, battery life can be significantly extended over devices requiring a microcontroller or other computing device. In other aspects, the apparatus 200 can comprise a microcontroller that is selectively powered to lower the power consumption of the apparatus.

In some aspects, the apparatus 200 can comprise a circuit 202. The circuit 202 can comprise a pressure sensing device 204 (e.g., a momentary switch SW1 in FIG. 6). The momentary switch SW1 can, when a force is applied thereto, close to permit current to flow through the circuit 202. Upon closing the circuit 202, current can flow to charge a capacitor C1. The charging rate of the capacitor C1 can be determined by a resistor R1. The resistor R1 can optionally be a potentiometer to permit adjustment of the charging rate. The circuit can comprise a transistor Q1 (e.g., a metal-oxide semiconductor field-effect transistor (MOSFET) that has a voltage threshold). (In other aspects, other switching devices can be used instead of the transistor Q1. For example, the switching device can be an analog switch integrated circuit (IC) or load switch IC. The load switch can, for example, toggle power to a motor or timer IC that, in turn, causes a vibratory motor to activate.) Once the voltage of the capacitor C1 charges to the voltage threshold, current can flow through the transistor Q1 to power an alarm M1. As illustrated, the alarm M1 can comprise or can be a vibratory eccentric motor. In other aspects, the alarm M1 can comprise an audible alarm, a visible alarm, or any other suitable alarm. Still further, the switchable current flow path of the transistor Q1 can be in communication with an integrated circuit timer (e.g., IC555 in a stable mode), a Bluetooth communication integrated circuit, a RF communication integrated circuit, or the like. Such devices, when positioned in communication with the switchable current flow path, can pulse the motor in a pattern or communicate with other devices. Thus, the alarm of the apparatus 200 can be configured in accordance with any of the alarms as disclosed herein. For example, the apparatus 200 can be configured to communicate with a remote computing device 60 (FIG. 4) or activate an audible alarm (e.g., a buzzer/speaker).

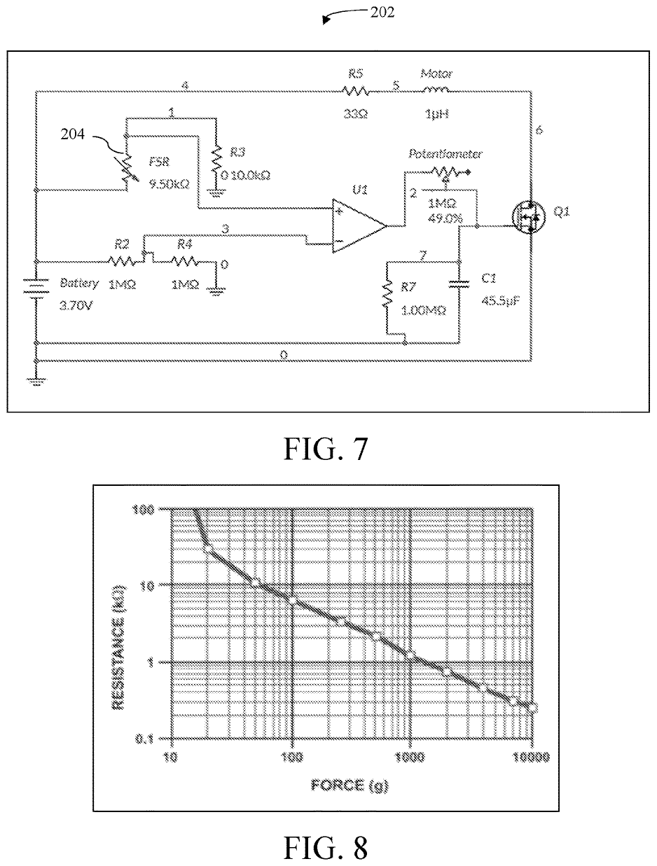

Non-limiting examples of the circuit 202 configuration and components are provided in FIGS. 6-7. The following list includes the labels associated with the various components of the circuit 202 depicted in FIG. 6.

### LIST OF COMPONENTS IN FIG. 6

VCC: Lithium-Ion Battery 3.7V
SW1: Physical Switch Used to Determine Force Application on Device
R1: Potentiometer Current Limiter for Time Delay. Increase Resistance Increase Delay
C1: Capacitor for Time Delay
R2: Bleed Resistor for Time Delay
Q1: Mosfet that Turns on Motor
M1: Vibratory Eccentric Motor
R3: Current Limiting Resistor for Motor

Optionally, the momentary switch 210 can have a have a threshold force required to actuate the momentary switch.

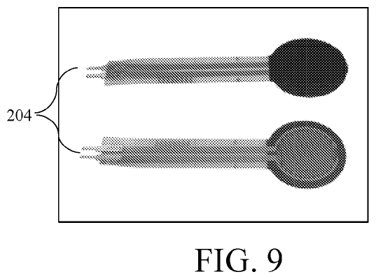

In further aspects, and with reference to FIG. 7, the circuit 202 can comprise a force-sensing resistor FSR that is configured to serve as a pressure sensing device 204 (FIG. 9) instead of the momentary switch M1 (FIG. 6). The force-sensing resistor 204 can have a variable resistance depending on pressure applied (FIG. 8). The force-sensing resistor 204 can be in electrical communication with a resistor (R3) so that the capacitor C1 begins to charge when the resistance of the force-sensing resistor 204 drops below the resistance R3. Thus, the resistance of resistor R3 can be selected in order to determine a threshold pressure that corresponds to a pressure that should trigger an alarm (e.g., sleeping on the force-sensing resistor 204). Optionally, the apparatus 200 can comprise a pad (e.g., a compressible pad) that influences the responsiveness of the FSR 204 or other pressure sensing device. The pad can comprise a material, such as conductive foam, conductive textile, or other carbon-doped rigid or elastomeric material, that can be placed on top of the underlying force-sensing resistor 204 to shape the pressure/resistance curve of the force-sensing resistor.

In some optional aspects, the force-sensing resistor 204 can be a force sensing resistor provided by Interlink Electronics (Camarillo, CA). In further optional aspects, the force-sensing resistor 204 can be integrally fabricated with the circuit 202. For example, the force-sensing resistor 204 can be integrated on a rigid substrate (e.g., a double-sided printed circuit board (PCB)). In these aspects, the force-sensing resistor 204 can form an outer portion of a housing of the apparatus 200. In this way, the durability of the apparatus 200 can be improved, and the profile of the apparatus can be reduced.

The following list includes the labels associated with the various components of the circuit 202 depicted in FIG. 7.

### LIST OF COMPONENTS IN FIG. 7

Battery: Lithium-Ion Battery 3.7V
R2 & R4: Voltage Divider for Comparator
FSR: Force Sensing Resistor R>10 MΩ When No Pressure Applied
R3: Resistor Chosen for Desired Force
U1: Rail to Rail Comparator
Potentiometer: Current Limiter for Time Delay. Increase Resistance Increase Delay
C1: Capacitor for Time Delay
R7: Bleed Resistor for Capacitor Used in Time Delay
Q1: Mosfet that Turns on Motor
Motor: Vibratory Eccentric Motor
R5: Current Limiting Resistor for Motor

In some aspects, and with reference to FIGS. 5-7, the apparatus 200 can comprise a circuit 202 comprising a pressure sensing device 204, a power source VCC; a transistor Q1 (or other switching device, such as, for example, an analog switch integrated circuit (IC) or load switch IC, as disclosed herein) having a voltage threshold and switchable flow path; and an alarm in communication with the switchable current flow path of the transistor. The transistor Q1 (or other switching device as disclosed herein) can be configured to permit current to flow to through a switchable current flow path upon receiving an applied voltage that exceeds the voltage threshold. The pressure sensing device, the power source, the transistor, and the alarm are electrically coupled so that closing of the momentary switch causes power to flow from the power source to the capacitor to charge the capacitor, wherein a voltage across the capacitor is configured to exceed the voltage threshold of the transistor after a predetermined time, thereby triggering the alarm.

The pressure sensing device can be, for example, a momentary switch or a force sensing resistor.

The circuit 202 can comprise a variable resistor (e.g., a potentiometer; R1 in FIG. 6; R7 in FIG. 7) in electrical communication with the capacitor C1. The variable resistor can be configured to permit adjustment of the predetermined time. The variable resistor can be electrically coupled in series with the capacitor.

In various aspects, the alarm can comprise a vibratory eccentric motor, a linear resonant actuator, a piezoelectric actuator, an audible alert, a visible alert, an electrical stimulation, or a combination thereof. In further or alternative aspects, the alarm can comprise one or both of a transmission to a remote device or an audible alarm. Accordingly, the alarm can comprise a vibration, a sound, a visible alert, an electrical stimulation, or a combination thereof.

In some optional aspects, the circuit 202 can comprise a microcontroller. For example, the microcontroller can be in communication with the switchable flow path of the switching device so that the microcontroller only receives power upon sufficient pressure applied to the pressure sensing device 204. In exemplary aspects, switchable current powering the microcontroller can occur after the RC delay circuit (comprising R1 and C1 in FIG. 6 or potentiometer R1 and capacitor C1 in FIG. 7). In other aspects, powering of the microcontroller can be driven directly by the output of the comparator U1 (in which case, the delay can be programed into the microcontroller). In this way, the circuit 202 can be lower power than an apparatus continually supplying power to the microcontroller. The microcontroller, once powered, can trigger any programmable action such as, for example, communicating with a remote computing device or triggering an alarm.

### Computing Device

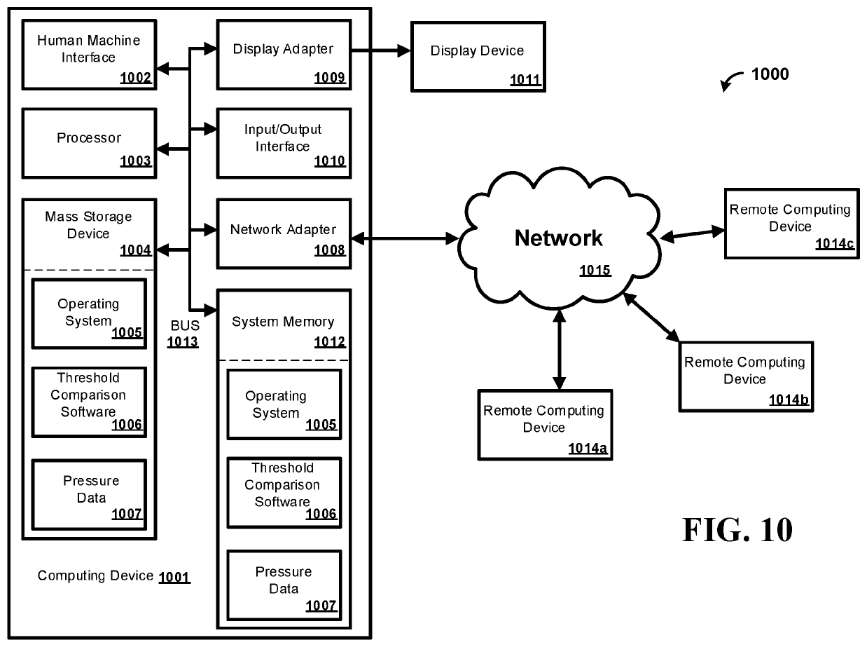

FIG. 10 shows an operating environment 1000 including an exemplary configuration of a computing device 1001 having architecture in accordance with the computing device 30 (FIG. 2) of the apparatus 10 (FIG. 2) or the remote computing device 60 (FIG. 4). In further exemplary aspects, a plurality of computing devices 30 (optionally, through separate remote computing devices 60) can be in communication with a central computing device, such as, for example, a server of a hospital or a computing device of a home with multiple device users).

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as pressure data 1007 (i.e., data from signals received by the pressure sensor(s)) and/or program modules such as operating system 1005 and threshold comparison software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and threshold comparison software 1006 may be stored on the mass storage device 1004. One or more of the operating system 1005 and threshold comparison software 1006 (or some combination thereof) may comprise program modules and the threshold comparison software 1006. The pressure data 1007 may also be stored on the mass storage device 1004. The pressure data 1007 may be stored in any of one or more databases known in the art. The databases may be centralized or distributed across multiple locations within the network 1015.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves and other body coverings, motion sensor, speech recognition, the pressure sensor 20 (FIG. 2) (e.g., a force sensitive resistor), combinations thereof, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices. For example, a graphic display can be provided on the remote computing device 60 (FIG. 4).

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. In various exemplary aspects, direct communication protocols such as radio frequency (RF) communication, LoRa communication, Bluetooth communication, or Bluetooth Low Energy (BTLE) communication can be used. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices.

Accordingly, at least a portion of the system 1000 can be configured with internet connectivity. It is contemplated that the remote computing device 1014a can be the remote computing device 60 (FIG. 6).

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: An apparatus comprising:
   a pressure sensor that is configured to generate pressure measurements indicative of pressure applied to the apparatus along a first axis; and
   a computing device in communication with the pressure sensor, the computing device comprising:
      a memory; and
      at least one processor in communication with the memory, wherein the memory comprises instructions that, when executed by the at least one processor, cause the at least one processor to:
         receive, from the pressure sensor, a plurality of pressure measurements comprising at least a first pressure measurement and a second pressure measurement, wherein each pressure measurement of the plurality of pressure measurements is spaced from each other pressure measurement of the plurality of pressure measurements by a predetermined duration;
         compare the first and second pressure measurements to a first predetermined pressure threshold; and
         cause, if the first and second pressure measurements exceed the first predetermined pressure threshold, an alarm.
Aspect 2: The apparatus of aspect 1, wherein the memory of the computing device comprises instructions that, when executed by the at least one processor, cause the at least one processor to:
   compare the first pressure measurement to a second predetermined pressure threshold, wherein the second predetermined pressure threshold is greater than the first predetermined pressure threshold and is indicative of an intentionally applied pressure; and
   initiate a function if the first measurement is greater than the second predetermined pressure threshold.
Aspect 3: The apparatus of aspect 2, wherein the function is a transition to stand-by mode.
Aspect 4: The apparatus of aspect 2 or aspect 3, wherein the memory of the computing device comprises instructions that, when executed by the at least one processor, cause the at least one processor to:
   compare the second pressure measurement to the second predetermined pressure threshold; and
   initiate the function only if the first and second pressure measurements are above the second predetermined pressure threshold.
Aspect 5: The apparatus of any one of the preceding aspects, wherein the memory of the computing device comprises instructions that, when executed by the at least one processor, cause the at least one processor to:
   cause the computing device to operate at a low power mode for an interval between receiving the first pressure measurement and receiving the second pressure measurement.
Aspect 6: The apparatus of aspect 5, wherein the interval is from about 5 seconds to about 30 seconds.
Aspect 7: The apparatus of any one of the preceding aspects, wherein the plurality of pressure measurements comprises at least one additional pressure measurement, wherein the memory of the computing device comprises instructions that, when executed by the at least one processor, cause the at least one processor to:
   receive each pressure measurement of the at least one additional pressure measurement from the pressure sensor;
   compare each pressure measurement of the at least one additional pressure measurement to the first predetermined pressure threshold;
   cause a counter to increase for each measurement of the plurality of pressure measurements that exceeds the first predetermined pressure threshold; and
   cause the alarm only if the count exceeds a count threshold.
Aspect 8: The apparatus of aspect 7, wherein exceeding the count threshold corresponds to a continued pressure above the first predetermined pressure threshold for a threshold duration.
Aspect 9: The apparatus of aspect 8, wherein the threshold duration is from about 30 seconds to about 2 minutes.
Aspect 10: The apparatus of any one of aspects 7-9, wherein the memory of the computing device comprises instructions that, when executed by the at least one processor, cause the at least one processor to:
   cause the counter not to increase for each measurement of the plurality of pressure measurements that exceeds the second predetermined pressure threshold.
Aspect 11: The apparatus of any one of aspects 7-10, wherein the memory of the computing device comprises instructions that, when executed by the at least one processor, cause the at least one processor to:
   reset the counter when each measurement of the plurality of measurements from the pressure sensor is below the first threshold.
Aspect 12: The apparatus of any one of the preceding aspects, further comprising an alarm device in communication with the computing device, wherein the alarm comprises operation of the alarm device.
Aspect 13: The apparatus of aspect 12, wherein the alarm device is a haptic alarm or electric stimulation.
Aspect 14: The apparatus of aspect 12, wherein the alarm device is an audible alarm.
Aspect 15: The apparatus of aspect 14, wherein the audible alarm comprises a voice recording.
Aspect 16: The apparatus of aspect 15, wherein the voice recording is comprises a voice familiar to the user.
Aspect 17: The apparatus of any one of the preceding aspects, further comprising a transmitter that is configured to communicate with a remote computing device.
Aspect 18: A system comprising:
   an apparatus of any one of the preceding aspects; and
   a continuous glucose monitor comprising a sensor that is configured to extend along a first axis into subcutaneous fatty tissue of a user, wherein the sensor is configured to determine a glucose measurement.
Aspect 19: The system of aspect 18, further comprising an insulin pump in communication with the glucose monitor, wherein the insulin pump is configured to deliver insulin based on glucose measurements received from the glucose sensor.
Aspect 20: The system of aspect 19, wherein the computing device is in communication with the insulin pump, wherein the memory of the computing device comprises instructions that, when executed by the at least one processor, cause the at least one processor to:
   adjust delivery of glucose by the insulin pump if the first pressure measurement or the second pressure measurement exceeds the first predetermined pressure threshold.
Aspect 21: The system of any one of aspects 18-20, wherein the pressure sensor comprises one of: a momentary switch, a barometric pressure sensor, a piezoelectric sensor, an accelerometer, a capacitive force sensor, a strain gauge, or a force-sensing resistor.
Aspect 22: The system of any one of aspects 18-21, wherein the pressure sensor is spaced laterally from the continuous glucose monitor along a lateral axis that is perpendicular to the first axis.
Aspect 23: The system of any one of aspects 18-22, wherein the pressure sensor is positioned above or below the continuous glucose monitor along the first axis.
Aspect 24: A system comprising:
   an apparatus as in aspect 17; and
   a remote computing device in communication with the transmitter.
Aspect 25: The system of aspect 24, wherein the remote computing device is a smartphone.
Aspect 26: The system of aspect 24 or aspect 25, wherein causing the alarm comprises causing the remote computing device to provide the alarm.
Aspect 27: The system of any one of aspects 24-26, wherein the alarm comprises: a vibration, a sound, a visible alert, an electrical stimulation, or a combination thereof.
Aspect 28: The system of aspect 27, wherein the alarm comprises the sound, wherein the sound comprises a voice recording of a voice familiar to the user.
Aspect 29: An apparatus comprising:
   a circuit comprising:
      a pressure sensing device;
      a power source;
      a switching device having a voltage threshold and switchable flow path, wherein the switching device is configured to permit current to flow to through a switchable current flow path upon receiving an applied voltage that exceeds the voltage threshold; and
      an alarm in communication with the switchable current flow path of the switching device,
      wherein the pressure sensing device, the power source, the switching device, and the alarm are electrically coupled so that closing of the momentary switch causes power to flow from the power source to the capacitor to charge the capacitor, wherein a voltage across the capacitor is configured to exceed the voltage threshold of the switching device after a predetermined time, thereby triggering the alarm.
Aspect 30: The apparatus of aspect 29, wherein the pressure sensing device is a momentary switch.
Aspect 31: The apparatus of aspect 29, wherein the pressure sensing device is a force sensing resistor.
Aspect 32: The apparatus of any one of aspects 29-31, further comprising a variable resistor in electrical communication with the capacitor, wherein the variable resistor is configured to permit adjustment of the predetermined time.
Aspect 33: The apparatus of aspect 32, wherein the variable resistor is a potentiometer electrically coupled in series with the capacitor.
Aspect 34: The apparatus of any one of aspects 29-33, wherein the alarm comprises a vibratory eccentric motor, a linear resonant actuator, a piezoelectric actuator, an audible alert, a visible alert, an electrical stimulation, or a combination thereof.
Aspect 35: The apparatus of any one of aspects 29-34, wherein the alarm comprises one of a transmission to a remote device or an audible alarm.

Those skilled in the art will recognize, or be able to ascertain using no more than routine experimentation, many equivalents to the specific embodiments of the method and compositions described herein. Such equivalents are intended to be encompassed by the following claims.

1. An apparatus comprising:
a pressure sensor that is configured to generate pressure measurements indicative of pressure applied to the apparatus along a first axis; and
a computing device in communication with the pressure sensor, the computing device comprising:
a memory; and
at least one processor in communication with the memory, wherein the memory comprises instructions that, when executed by the at least one processor, cause the at least one processor to:
receive, from the pressure sensor, a plurality of pressure measurements comprising at least a first pressure measurement and a second pressure measurement, wherein each pressure measurement of the plurality of pressure measurements is spaced from each other pressure measurement of the plurality of pressure measurements by a predetermined duration;
compare the first and second pressure measurements to a first predetermined pressure threshold; and
cause, if the first and second pressure measurements exceed the first predetermined pressure threshold, an alarm.

2. The apparatus of claim 1, wherein the memory of the computing device comprises instructions that, when executed by the at least one processor, cause the at least one processor to:
compare the first pressure measurement to a second predetermined pressure threshold, wherein the second predetermined pressure threshold is greater than the first predetermined pressure threshold and is indicative of an intentionally applied pressure; and
initiate a function if the first measurement is greater than the second predetermined pressure threshold.

3. The apparatus of claim 2, wherein the function is a transition to stand-by mode.

4. The apparatus of claim 2, wherein the memory of the computing device comprises instructions that, when executed by the at least one processor, cause the at least one processor to:
compare the second pressure measurement to the second predetermined pressure threshold; and
initiate the function only if the first and second pressure measurements are above the second predetermined pressure threshold.

5. The apparatus of claim 1, wherein the memory of the computing device comprises instructions that, when executed by the at least one processor, cause the at least one processor to:
cause the computing device to operate at a low power mode for an interval between receiving the first pressure measurement and receiving the second pressure measurement.

6. (canceled)

7. The apparatus of claim 1, wherein the plurality of pressure measurements comprises at least one additional pressure measurement, wherein the memory of the computing device comprises instructions that, when executed by the at least one processor, cause the at least one processor to:
receive each pressure measurement of the at least one additional pressure measurement from the pressure sensor;
compare each pressure measurement of the at least one additional pressure measurement to the first predetermined pressure threshold;
cause a counter to increase for each measurement of the plurality of pressure measurements that exceeds the first predetermined pressure threshold; and
cause the alarm only if the count exceeds a count threshold.

8. The apparatus of claim 7, wherein exceeding the count threshold corresponds to a continued pressure above the first predetermined pressure threshold for a threshold duration.

9. (canceled)

10. The apparatus of claim 7, wherein the memory of the computing device comprises instructions that, when executed by the at least one processor, cause the at least one processor to:
cause the counter not to increase for each measurement of the plurality of pressure measurements that exceeds the second predetermined pressure threshold.

11. The apparatus of claim 7, wherein the memory of the computing device comprises instructions that, when executed by the at least one processor, cause the at least one processor to:
reset the counter when each measurement of the plurality of measurements from the pressure sensor is below the first threshold.

12. The apparatus of claim 1, further comprising an alarm device in communication with the computing device, wherein the alarm comprises operation of the alarm device, and wherein the alarm is a haptic alarm, electric stimulation, or an audible alarm.

13. (canceled)

14. (canceled)

15. (canceled)

16. (canceled)

17. The apparatus of claim 1, further comprising a transmitter that is configured to communicate with a remote computing device.

18. A system comprising:
the apparatus of claim 1; and
a continuous glucose monitor comprising a sensor that is configured to extend along a first axis into subcutaneous fatty tissue of a user, wherein the sensor is configured to determine a glucose measurement.

19. The system of claim 18, further comprising an insulin pump in communication with the glucose monitor, wherein the insulin pump is configured to deliver insulin based on glucose measurements received from the glucose sensor.

20. The system of claim 19, wherein the computing device is in communication with the insulin pump, wherein the memory of the computing device comprises instructions that, when executed by the at least one processor, cause the at least one processor to:
adjust delivery of glucose by the insulin pump if the first pressure measurement or the second pressure measurement exceeds the first predetermined pressure threshold.

21. The system of claim 18, wherein the pressure sensor comprises one of: a momentary switch, a barometric pressure sensor, a piezoelectric sensor, an accelerometer, a capacitive force sensor, a strain gauge, or a force-sensing resistor.

22. The system of claim 18, wherein the pressure sensor is spaced laterally from the continuous glucose monitor along a lateral axis that is perpendicular to the first axis.

23. The system of claim 18, wherein the pressure sensor is positioned above or below the continuous glucose monitor along the first axis.

24. A system comprising:
The apparatus as in claim 17; and
a remote computing device in communication with the transmitter.
wherein causing the alarm comprises causing the remote computing device to provide the alarm, and
wherein the alarm comprises: a vibration, a sound, a visible alert, an electrical stimulation, or a combination thereof.

25. (canceled)

26. (canceled)

27. (canceled)

28. (canceled)

29. An apparatus comprising:
a circuit comprising:
a pressure sensing device;
a power source;
a switching device having a voltage threshold and switchable flow path, wherein the switching device is configured to permit current to flow to through a switchable current flow path upon receiving an applied voltage that exceeds the voltage threshold; and
an alarm in communication with the switchable current flow path of the switching device,
wherein the pressure sensing device, the power source, the switching device, and the alarm are electrically coupled so that closing of the momentary switch causes power to flow from the power source to the capacitor to charge the capacitor, wherein a voltage across the capacitor is configured to exceed the voltage threshold of the switching device after a predetermined time, thereby triggering the alarm.

30. (canceled)

31. (canceled)

32. The apparatus of claim 29, further comprising a variable resistor in electrical communication with the capacitor, wherein the variable resistor is configured to permit adjustment of the predetermined time.

33. (canceled)

34. (canceled)

35. (canceled)
